(19) United States
(12) Patent Application Publication
(10) Pub. No.: US 2026/0171188 A1
(43) Pub. Date: Jun. 18, 2026

(54) MACHINE LEARNING FOR ANTIBODY DISCOVERY AND USES THEREOF

(71) Applicants: ZHEJIANG NANOMAB TECHNOLOGY CENTER CO. LTD., SHAOXING, ZHEJIANG (CN); SHANGHAI CHENGHUANG NANOMAB TECHNOLOGY CO. LTD., Jiading District, Shanghai (CN)

(72) Inventors: Xinhao WANG, Shanghai (CN); Yu CHEN, Shanghai (CN); Weimin ZHU, Shanghai (CN)

(21) Appl. No.: 19/127,090

(22) PCT Filed: Nov. 2, 2023
(86) PCT No.: PCT/CN2023/129238

§ 371 Date: May 2, 2025

Related U.S. Application Data
(60) Provisional application No. 63/382,101, filed on Nov. 2, 2022.

(51) Int. Cl. G16B 15/30; G16B 30/10; G16B 40/20

(52) U.S. Cl. CPC G16B 15/30; G16B 30/10; G16B 40/20

(57) ABSTRACT

Provided is a method for identifying an antibody that has a certain biological function in relation to an antigen using a machine learning model. The method comprises the steps of: a) obtaining antibodies from B cells of at least one animal immunized with the antigen; b) determining the sequences of the antibodies in a) or fragments thereof and at least one type of functional data thereof; c) building a machine learning model using one or more machine learning algorithms, and training the model using training data, wherein the training data comprises the sequences and functional data in b); d) using the trained model to predict the ability of sequences from B cells of the immunized animal in a) or from B cells of a different animal immunized with the antigen, to encode an antibody that has the biological function in relation to the antigen; e) generating one or more antibodies from the sequences in d) predicted to encode an antibody that has the biological function in relation to the antigen; and f) determining whether the antibodies in e) has the predicted biological function. Enrichment scores of the sequences, CDR groups, lineages and/or clusters of the selected sequences in step d) are calculated and those having a higher enrichment score are selected to generate the antibodies.

### CROSS-REFERENCE TO RELATED APPLICATIONS

This application claims the benefit of U.S. provisional application No. 63/382,101, filed Nov. 2, 2022, which is incorporated by reference in its entirety.

### BACKGROUND OF THE INVENTION

### (1) Field of the Invention

The present application generally relates to identification of antibodies. More specifically, the application uses machine learning prediction as another criterion in sequence selection and applies it iteratively for antibody discovery from libraries of sequences encoding antibodies.

### (2) Description of the Related Art

To become a drug, an antibody has to satisfy many criteria (functionality, immunogenicity, developability, etc.). Many antibodies will fail during development process. So for therapeutic antibody drug development, it is very critical to identify as many high affinity binders from repertoire as possible in order to have a large pool of antibodies to choose from for clinical development. In addition, since the quality of an antibody (high affinity, specificity, low immunogenicity, etc.) has a direct impact on final drug efficacy, having a large pool of binders is more likely to lead to discovery of high quality binders.

The natural immune-repertoire exhibits a power-law distribution of its clones: high count clones are very few and many different clones have low counts (FIGS. 1A, 1B). Because of such a distribution, traditional screening methods using phage display, hybridoma or B cell panning technologies are not efficient at identifying low count clones with limited sampling depth. Traditional screening methods enable people to find high affinity binders in 10-15% repertoire space with around 10 plates (˜1000 clones) (FIG. 1A, 1B). High throughput sequencing technology such as next generation sequencing (NGS) technology, on the other hand, can sequence millions of clones in a cost-effective manner. Its sampling depth is more than 3 orders of magnitude higher than traditional screening method with 10 plates. With such sampling depth, a higher diversity of clones including many rare clones are expected to be captured as sequences from repertoire (Deschaght P et al., 2017). The challenge becomes how to effectively identify highly desirable clones from millions of sequences.

During phage library screening steps, the diversity of repertoire is reduced and clones are enriched with each rounds of panning and expansion. By comparing the frequency of clones in repertoire before and after each round of panning, enrichment score can be calculated. In principle, if the only factor affecting the panning is affinity, then enrichment score will be a good metric for selecting clones with high affinity. However, panning and expansion are affected by many factors, including antigen accessibility, antibody affinity, antibody display on phage and phage amplification in bacteria (Ljungars A et al., 2019). From our own experience and others, enrichment score alone is not effective in selecting high affinity clones from sequence data.

Multiple physical-biochemical properties (CDR charge, hydropathy, CDR3 length, CDR conformation etc.) of an antibody contribute to the affinity and specificity of an antibody, and different epitopes may require different combinations of the above properties to achieve high affinity and specificity. Machine learning models, especially deep learning models, are very good at integrating multiple factors with non-linear relationships and predicting results with high accuracy. The application of such methods has been successfully demonstrated in many fields including biomedicine (Narayanan et al., 2021).

Machine learning has been explored in specific scenarios for antibody design and discovery. See, e.g., US Patent Application Publication 2019/0065677A1 and PCT Patent Publication WO 2020/208555A1, both of which are incorporated by reference. In both applications, tens of thousands training data generated experimentally using certain quality metrics as proxy for certain antibody characters, like affinity, were used to train deep learning models. Trained deep learning models were then used to generate novel antibody sequences for testing or to predict the characters of in silico generated antibody sequences. However, such approach has not gained much practical use for discovering novel antibodies against a specific target, as there is little incentive to discover more novel binders when there are a large number of binders available already but yet to be characterized. In this application, we used hundreds of experimentally determined functional data measured by ELISA (enzyme-linked immunosorbent), FACS (fluorescence-activated cell sorting) or other means as training data to train deep learning models, and the trained models are then used to predict the biological functions (for example, affinity) of clones from NGS sequences. To improve prediction robustness with smaller dataset, machine learning techniques such as pre-training and transfer learning can also be utilized in the process (Leem et al., 2022; Biswas et al., 2021). Prediction results are then combined with enrichment information of the clone and others as criteria to select clones from NGS sequences for antibody discovery.

### BRIEF SUMMARY OF THE INVENTION

The present invention is based in part on the discovery of an effective method for identifying antibodies that has certain biological function in relation to an antigen using a machine learning model and optionally in combination with enrichment score calculation. The method utilized the trained model to evaluate antibody sequences encoded in libraries of sequences from B cells from animals immunized with the antigen.

Thus, in some embodiments, provided is a method for identifying an antibody that has a preferred biological function in relation to an antigen. In these embodiments, the method comprises:
a) obtaining antibodies from B cells of at least one animal immunized with the antigen;
b) determining the sequences of the antibodies in a) or fragments thereof and at least one type of functional data thereof;
c) building a machine learning model using one or more machine learning algorithms, and training the model using training data, wherein the training data comprises the sequences and functional data in b);
d) using the trained model to predict the ability of sequences from B cells of the immunized animal in a) or from B cells of a different animal immunized with the antigen, to encode an antibody that has the biological function in relation to the antigen;
e) generating one or more antibodies from the sequences in d) predicted to encode an antibody that has the biological function in relation to the antigen; and
f) determining whether the antibodies in e) has the biological function.

In some embodiments, the biological function in relation to the antigen is specific binding, neutralization or potentiation. In some embodiments, the sequences predicted by the trained machine learning model to encode an antibody that has the biological function in relation to the antigen are grouped by CDR groups, lineages and/or clusters, and enrichment scores of such sequences, CDR groups, lineages and/or clusters are calculated. Antibodies from the sequences predicted to encode an antibody that has the biological function in relation to the antigen and having a high enrichment score of the sequences, CDR groups, lineages and/or clusters are generated and their predicted biological function in relation to the antigen is then verified.

### BRIEF DESCRIPTION OF THE SEVERAL VIEWS OF THE DRAWINGS

FIGS. 1A and 1B are graphs showing the distribution of antibody clones by number of sequences, CDR3 sequence length and count.

FIG. 2 is a flow chart showing exemplary steps for applying machine learning methods for antibody discovery.

FIG. 3 is a flow chart showing exemplary data processing steps for NGS sequences generated by Miseq.

FIG. 4 is an example of a one hot encoding for antibody discovery.

FIG. 5 is a flow chart showing an exemplary machine learning model for antibody discovery.

FIG. 6 is a flow chart showing another exemplary machine learning model for antibody discovery.

FIG. 7 is a flow chart showing an exemplary machine learning model using both antibody and antigen sequences as inputs for antibody discovery.

FIG. 8 is a flow chart showing an exemplary process using pre-training/transfer learning (FIG. 8A) and supervised learning (FIG. 8B) for antibody discovery.

FIG. 9 is a graph showing the distribution of normalized ELISA values in training data.

FIG. 10 shows an example of results from ELISA testing of antibody clones selected based on machine learning.

FIG. 11 shows results of a blocking assay of clones selected based on machine learning.

FIG. 12 shows lineage distribution of clones selected based on machine learning and corresponding positive rate.

FIG. 13 shows prediction performances of two algorithms with two sequence representations based on area under curve (AUC) value.

FIG. 14 shows prediction performances of 13 algorithms with ESM2 sequence representation based on area under curve (AUC) value.

FIG. 15 shows ELISA binding data for 44 tested clones.

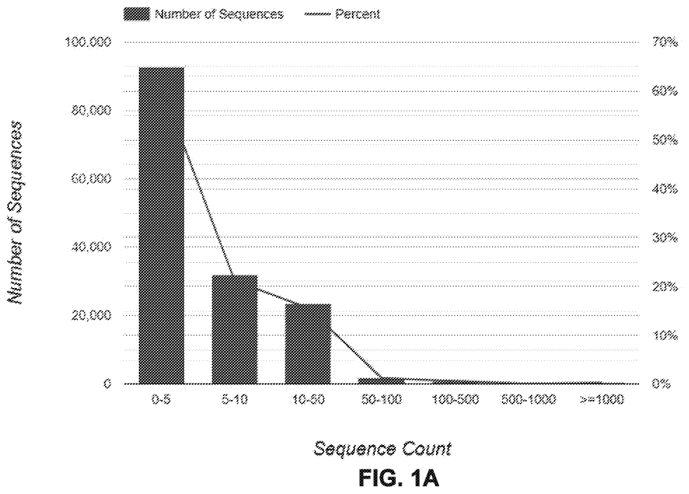

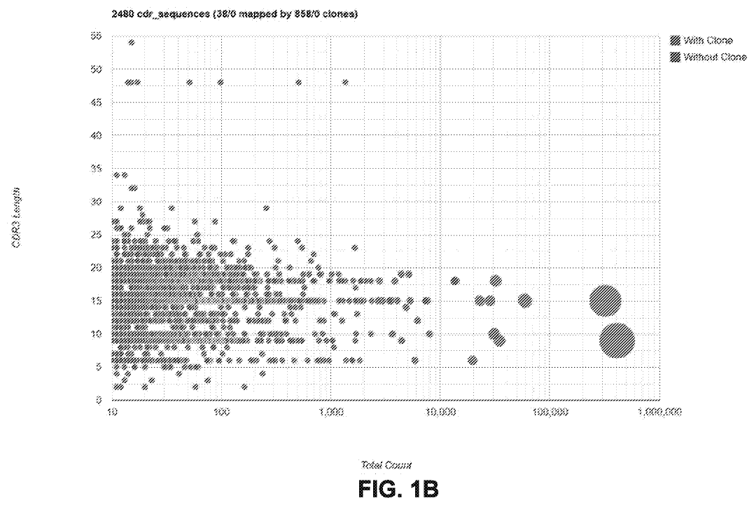

### DETAILED DESCRIPTION OF THE INVENTION

### Definitions

The term “plurality” refers to more than 1, for example more than 2, more than about 5, more than about 10, more than about 20, more than about 50, more than about 100, more than about 200, more than about 500, more than about 1000, more than about 2000, more than about 5000, more than about 10,000, more than about 20,000, more than about 50,000, more than about 100,000, usually no more than about 200,000. A “population” contains a plurality of items.

The term “epitope” as used herein can include any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the equilibrium dissociation constant is ≤1 μM, preferably ≤100 nM and most preferably ≤10 nM.

The term “K_{D}” refers to the equilibrium dissociation constant of a particular antibody-antigen interaction.

The term “immune response” as used herein can refer to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from an organism of invading pathogens, cells or tissues infected with pathogens, cancerous cells, or, in cases of autoimmunity or pathological inflammation, normal organismal cells or tissues.

As used herein, the term “antibody” refers to (a) an intact immunoglobulin, (b) a monoclonal or polyclonal antigen-binding fragment with the Fc (crystallizable fragment) region or FcRn binding fragment of the Fc region (“Fc fragment” or “Fc region”), (c) a nanobody (including naturally occurring camelid nanobodies and heavy chain only [“VHH”] antibodies), or (d) an IgNAR antibody found in sharks and other elasmobranchs. The antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single region antibodies, chimeric antibodies, CDR grafted antibodies, humanized antibodies, biparatopic antibodies, diabodies and polypeptides that contain at least a portion of an immunoglobulin that is sufficient to confer specific antigen binding to the polypeptide. The Fe region includes portions of two heavy chains contributing to two or three classes of the antibody. The Fc region may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 regions; (ii) the Fab′ fragment, which is a Fab fragment having one or more cysteine residues at the C-terminus of the CH1 region; (iii) the Fd fragment having VH and CH1 regions; (iv) the Fd′ fragment having VH and CH1 regions and one or more cysteine residues at the C-terminus of the CH1 region; (v) the Fv fragment having the VL and VH regions of a single arm of an antibody; (vi) the dAb fragment (Ward et al., 1989) which consists of a VH region; (vii) isolated CDR regions; (viii) F(ab′)2 fragments, a bivalent fragment including two Fab′ fragments linked by a disulfide bridge at the hinge region; (ix) single chain antibody molecules (e.g., single chain Fv; scFv) (Bird et al., 1988; Huston et al., 1988); (x) “diabodies” with two antigen binding sites, comprising a heavy chain variable region (VH) connected to a light chain variable region (VL) in the same polypeptide chain (see, e.g., EP 404,097; WO 93/11161; Hollinger et al., 1993); (xi) “linear antibodies” comprising a pair of tandem Fd segments (VH-CH1-VH-CH1) which, together with complementary light chain polypeptides, form a pair of antigen binding regions (Zapata et al., 1995; U.S. Pat. No. 5,641,870.

“Single-chain variable fragment”, “single-chain antibody variable fragments” or “scFv” antibodies as used herein refers to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chains may be in any order, for example, VH-linker-VL or VL-linker-VH, so long as the specificity of the scFv to the target antigen is retained.

An “isolated antibody”, as used herein, can refer to an antibody that is substantially free of other antibodies having different antigenic specificities (e.g., an isolated antibody that specifically binds a TRAIL protein can be substantially free of antibodies that specifically bind antigens other than TRAIL proteins). An isolated antibody that specifically binds a human TRAIL protein can, however, have cross-reactivity to other antigens, such as TRAIL proteins from other species. Moreover, an isolated antibody can be substantially free of other cellular material and/or chemicals.

The terms “monoclonal antibody” or “monoclonal antibody composition” as used herein can refer to a preparation of antibody molecules of single molecular composition. A monoclonal antibody composition displays a single binding specificity and affinity for a particular epitope.

The term “recombinant human antibody”, as used herein, can refer to all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” can refer to the antibody class (e.g., IgM or IgG1) that is encoded by the heavy chain constant region genes. An antibody can be an immunoglobulin G (IgG), an IgM, an IgE, an IgA or an IgD molecule, or is derived therefrom.

The term “VHH²”, “VHH³” and “VH¹” are representing the heavy chains of three camelid IgG isotypes IgG2, IgG3 and IgG1 respectively. VL¹ is representing the light chain of camelid IgG1. Camelid VL¹ includes, but not limited to Vκ and Vλ.

The term “correspondingly positioned amino acids” and “corresponding amino acids” used herein interchangeably, are amino acid residues that are at an identical position (i.e., they lie across from each other) When two or more amino acid sequences are aligned. Methods for aligning and numbering antibody sequences are well known in the art.

The term “natural” antibody refers to an antibody in which the heavy and light chains of the antibody have been made and paired by the immune system of a multicellular organism. Spleen, lymph nodes, bone marrow, blood and other lymphatic tissues are examples of tissues that contain cells that produce natural antibodies. For example, the antibodies produced by B cells isolated from a first animal immunized with an antigen are natural antibodies. Natural antibodies contain naturally-paired heavy and light chains.

The term “naturally paired” refers to heavy and light chain sequences that have been paired by the immune system of a multi-cellular organism.

The term “mixture”, as used herein, refers to a combination of elements, e. g., cells, that are interspersed and not in any particular order. A mixture is homogeneous and not spatially separated into its different constituents. Examples of mixtures of elements include a number of different cells that are present in the same aqueous solution in a spatially undressed manner.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “enriched” is intended to refer to component of a composition (e.g., a particular type of cells) that is more concentrated (e.g., at least 2×, at least 5×, at least 10×, at least 50×, at least 100×, at least 500×, at least 1,000×), relative to other components in the sample (e.g., other cells) than prior to enrichment. In some cases, something that is enriched may represent a significant percent (e.g., greater than 2%, greater than 5%, greater than 10%, greater than 20%, greater than 50%, or more, usually up to about 90%-100%) of the sample in which it resides.

The term “enriching” is intended to any way by which antigen-specific cells can be obtained from a larger population of B cells. As described in greater detail below, enriching may be done by panning, using a bead or cell sorting, for example.

The term “enrichment score” refers to the metric measuring the enrichment of sequences and groups. It is calculated from the frequencies of sequence, CDR/lineage/cluster groups in one library divided by the corresponding frequencies in its reference library. High enrichment score has minimum score value of 2.

The term “reference library” for a library A refers to the library from samples before any specific action which produced the sample for library A. If there is only one round panning, the reference library is the library derived from pre-panning samples. If there are two or more rounds of panning, then the reference library for the library from specific round of panning is the one from previous round of panning. If there is no panning, but with immunization, the reference library for a library is the one from sample before immunization.

The term “obtaining” in the context of obtaining an element, e. g., cells or sequences, is intended to include receiving the element as well as physically producing the element.

A cell is “derived from” a host if the cell, or the progeny thereof, was obtained from the host. The progeny of a progenitor cell is derived from the progenitor cell.

The term “panning” is used to refer to a method by which B cells are applied to a container (e.g., a plate) that has one or more surfaces that are coated in an antigen or portion thereof. Unbound cells can be removed by washing the surface after the cells are applied to it.

The term “bead-based enrichment” is used to refer to a method by which B cells are mixed with beads, e.g., magnetic beads, that are linked to an antigen or portion thereof.

The term “cell sorting” is used to refer to a method by which B cells are mixed a detectable antigen (e.g., a fluorescently detectable antigen) in solution. In cell sorting methods, cells that are bound to the antigen are sorted from the unbound cells. Fluorescence-activated cell sorting (FACS) is an example of a cell sorting method.

The term “activating” is referred to the stimulation of B cells to a) proliferate and b) differentiate into plasma blasts and/or plasma cells and c) secrete antibodies. B cell activation can be done by contacting the B cells with antigen, T cells expressing CD40L and cytokines, although other methods are known (see, e.g., Wykes, Imm. Cell. Biol. 2003 81: 328-331).

The term “activated B cells” refers to a cell population that comprises the progeny of a B cell that was activated. As noted above, activation causes B cells to proliferate, and the progeny of such cells are referred to herein as activated B cells.

The term “collecting” refers to the act of separating the cells that in the culture medium from a substrate. Collecting may be done by pipetting or by decanting, for example.

The term “immunized by an antigen” and grammatical equivalents thereof (e.g., “immunized animal”) is intended to refer to any animal (humans, rabbits, mice, rats, sheep, cows, chickens, camels) that is mounting an immune response an antigen. An animal may be exposed to a foreign antigen via exposure to an infectious agent, a vaccination, or by administrating an antigen and adjuvant (e.g., by injection), for example. The term “immunized by an antigen” is also intended to include animals that are mounting an immune response against a “self” antigen, i.e., have an autoimmune disease.

The term “lineage rank” refers to the order of lineages when they are listed by their priority factors. The priority factors include but not limited to abundancy of lineage sequences, amplification factor, dynamic change of lineage sequence before and after depleting certain unwanted B cells, dynamic change of lineage sequence abundancy during immunization course, lineages which share the same naïve B-cell origin between VHH and VH, avoidance of developability liability sequences and a combination thereof.

The term “hamming distance” refers to the number of positions at which the corresponding symbols are different between two sequences of equal length.

As used herein, the term “grouped antibodies by lineage”, “lineage-related antibodies” and “antibodies that related by lineage” as well as grammatically-equivalent variants thereof, are antibodies that are produced by cells that share a common B cell ancestor. Antibodies that are related by lineage bind to the same epitope of an antigen and are typically very similar in sequence, particularly in their light chain and heavy chain CDR3s. Both the heavy chain and light chain CDR3s of lineage-related antibodies can have an identical length and a near identical sequence (i.e., differ by up to 5, i.e., 0, 1, 2, 3, 4 or 5 residues). Among the group of CDR3s from a lineage, minimal CDR3 distance of a specific CDR3 is the smallest hamming distance of this CDR3 comparing with all other CDR3 of the same length. In some embodiments, the minimal CDR3 distance is equal to or less than 1. In certain cases, the B cell ancestor contains a genome having a rearranged light chain VIC region and a rearranged heavy chain VDJ region, and produces an antibody that has not yet undergone affinity maturation. “Naïve” or “virgin” B cells present in spleen tissue, are exemplary B cell common ancestors.

Related antibodies are related via a common antibody ancestor, e.g., the antibody produced in the naïve B cell ancestor. The term “lineage related antibodies” is intended to describe a group of antibodies that are produced by cells that arise from the same ancestor B-cell. A “lineage group” contains a group of antibodies that are related to one another by lineage.

As used herein, the term “at least the CDR3s” or “at least the CDR3 sequences” refers to only CDR3 sequences, CDR3 sequences in conjunction with CDR1 and/or CDR2 sequences or a sequence of at least 50 contiguous amino acids of the variable domain, up to the entire length of the variable domain, where the sequence contains a CDR3 sequence.

As used herein, the terms “lineage tree” refers to a diagram, resulting from a cladistics analysis, which depicts a hypothetical branching sequence of lineages leading to the individual species of interest. The points of branching within a lineage tree are called nodes.

As used herein, the term “lineage” refers to a theoretical line of descent. Sometimes a group of antibodies related by lineage is referred to as a “lineage group”. The term “lineage” is exclusive, in that a sequence can belong to only one lineage.

As used herein, the term “subgrouping” refers to a further grouping of sequences in a lineage based on unique features or signatures. “Subgroup” is not exclusive, which means one sequence can be in different subgroups. For example, one sequence can have two, three, four, five, or six unique features at the same time. Applying sequence signatures can help to select/narrow-down testing lineages (representative sequences) in a better manner, which may have better biological function/bioactivity outcomes.

As used herein, the term “lineage analysis” refers to the analysis of the theoretical line of descent of an antibody, which is usually done by analyzing a lineage tree.

As used herein, the term “sequence read” refers to a sequence of nucleotides determined by a sequencer, which determination is made, for example, by means of base calling software associated with the technique.

As used herein, the term “obtaining the amino acid sequences” refers to obtaining a file containing amino acid sequences. As is well known, a nucleic acid sequence can be translated into an amino acid sequence in silico.

The term “anchor” and “anchor binder” as used herein interchangeably, is referred to conventional antibody generated with single B-cells sorting or heterohybridoma having native H and L pairing, with that, ones can “position/pair” heavy chain lineage and light chain lineage which consist of a group of sequences derived from clonal expansion of naïve B-cell H and L sequences after encountering the epitope of antigen. Lineages can be “anchored” considering the amino acid sequences of heavy and light chains that are known to pair with one another. In these embodiments, the branches are rotated around their nodes until there is a minimal number of cross-overs (e.g., no crossovers) between the anchored sequences. After the trees have been “aligned” by tanglegram analysis, the leaves that are known to pair can be connected by an edge. If the leaves that are known to pair are connected by an edge, the intervening leaves, in theory, can pair with one another as long as they do not create a cross-over event with an edge or one another.

The phrases “a monoclonal antibody recognizing an epitope on the antigen”, “an antibody recognizing an antigen” and “an antibody specific for an antigen” are used interchangeably herein with the term “an antibody which binds specifically to an antigen” or grammatical equivalents thereof.

The term “specific binding” refers to the ability of an antibody to preferentially bind to a particular antigen that is present in a homogeneous mixture of different molecules. In certain embodiments, a specific binding interaction will discriminate between desirable and undesirable molecules in a sample, in some embodiments more than about 10 to 100 fold or more than e.g., about 1000- or 10,000 fold.

The term “does not substantially bind” to a protein or cells, as used herein, can mean that it cannot bind or does not bind with a high affinity to the protein or cells, i.e., binds to the protein or cells with an K_{D} of 2×10⁻⁶ M or more, more preferably 1×10⁻⁵ M or more, more preferably 1×10⁻⁴ M or more, more preferably 1×10⁻³ M or more, even more preferably 1×10⁻² M or more.

The term “high affinity” for an IgG antibody can refer to an antibody having a K_{D} of 1×10⁻⁶ M or less, preferably 1×10⁻⁷ M or less, more preferably 1×10⁻⁸ M or less, even more preferably 1×10⁻⁹ M or less, even more preferably 1×10⁻⁰M or less for a target antigen. However, “high affinity” binding can vary for other antibody isotypes.

A “CDR grafted antibody” is an antibody comprising one or more CDRs derived from an antibody of a particular species or isotype and the framework of another antibody of the same or different species or isotype.

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant regions of the heavy and/or light chains of the non-human species antibody are mutated to produce the humanized antibody. In another embodiment, the constant region(s) from a human antibody are fused to the variable region(s) of a non-human species. In another embodiment, a humanized antibody is a CDR grafted antibody comprising one or more CDRs derived from an antibody of a particular species or isotype and the framework of human antibodies. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. Other combinations are possible.

The term “biparatopic antibody” refers to an antibody binds to two non-overlapping epitopes of an antigen. In some embodiments, the biparatopic antibody comprises heavy chain only VHHs without light chain. In some embodiments, the biparatopic antibody comprises both heavy chain only VHHs and conventional VH¹/VL¹ pairs. In some embodiments, the biparatopic antibody comprises two conventional VH¹/VL¹ pairs. In some embodiments, the biparatopic antibody has a first heavy chain and a first light chain from a monoclonal antibody targeting one epitope, and an additional antibody heavy chain and light chain targeting another epitope. In some embodiments, the additional light chain or heavy chain can be different from the first light or heavy chains. The binding of an antibody of the disclosed invention to an antigen can be assessed using one or more techniques well established in the art. For example, in some embodiments, an antibody is tested by ELISA assays, for example using a recombinant antigen protein. Still other suitable binding assays include but are not limited to a flow cytometry assay in which the antibody is reacted with a cell line that expresses the human antigen, such as HEK293 cells. Additionally or alternatively, the binding of the antibody, including the binding kinetics (e.g., K_{D} value) can be tested in BIAcore binding assays, Octet Red96 (Pall) and the like.

The term “single B-cell sorting” refers to the sorting of isolated and separated single B cells based on antigen specificity. Technologies for single-cell separation, isolation, and sorting include but are not limited to: FACS (fluorescent activated cell sorting, e.g. using a fluorescent-tagged antigen to isolate cells that bind the antigen), ISAAC (immunospot array assays on a chip), LCM (laser-capture microdissection), microengraving, and droplet microfluidics.

The term “ELISA OD value” refers to the optical density measured in enzyme-linked immunosorbent (ELISA) assays. In antibody assays, its value depends on antigen/antibody concentration and binding affinity.

The term “cross validation” refers to the statistical technique used to test the effectiveness of a machine learning model. In a “10-fold cross validation”, the fitting procedure is applied ten times, with each being performed on 90% of the total training data selected at random, with the remaining 10% used for validation.

The term “supervised learning” or “supervised machine learning” refers to a subcategory of machine learning and artificial intelligence, which uses labeled datasets to train algorithms to classify data or predict outcomes accurately.

The term “unsupervised learning” or “unsupervised machine learning” refers to a subcategory of machine learning and artificial intelligence, which uses unlabeled dataset to train algorithms to analyze data and to find hidden patterns and insights in the data without the need for human intervention.

Provided is a method of generating an antibody that has a certain biological activity in relation to an antigen. In these embodiments, the method comprises the steps of
a) obtaining antibodies from B cells of at least one animal immunized with the antigen;
b) determining the sequences of the antibodies in a) or fragments thereof and at least one type of functional data thereof;
c) building a machine learning model using one or more machine learning algorithms, and training the model using training data, wherein the training data comprises the sequences and functional data in b);
d) using the trained model to predict the ability of sequences from B cells of the immunized animal in a) or from B cells of a different animal immunized with the antigen, to encode an antibody that has the biological function in relation to the antigen;
e) generating one or more antibodies from the sequences in d) predicted to encode an antibody that has the biological function in relation to the antigen; and
f) determining whether the antibodies in e) has the biological function.

For these methods, the library of antibody sequences can be created by any means now known or later discovered. In some embodiments, the library is sequenced by next generation sequencing (NGS) methods known in the art. Any NGS method can be utilized in these embodiments. See, e.g., Slatko et al. (2018) for an overview of NGS methods.

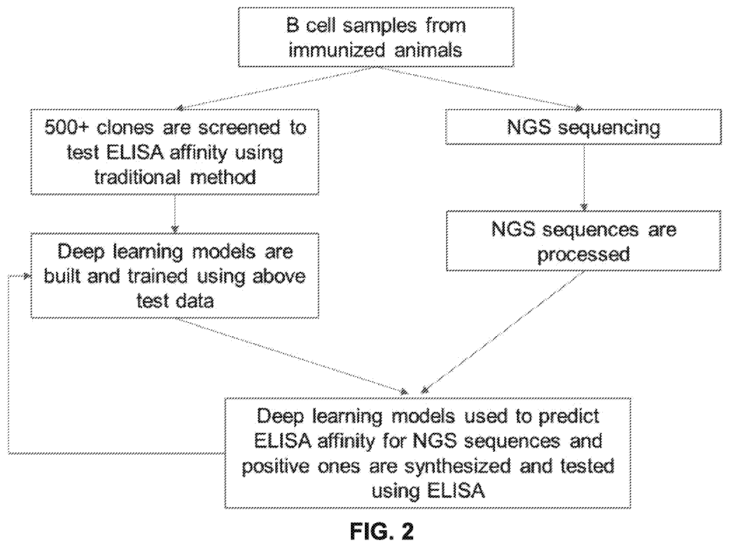

FIG. 2 shows exemplary steps for creating the machine learning model. To generate training data, traditional screen methods like phage display can be used to generate 500+ clones with ELISA data from B cell samples of immunized animals. The model is then built and trained with these data. Trained machine learning model is then used to predict the sequences from NGS data, which is generated from the same B cell samples. Positive predicted sequences can be synthesized.

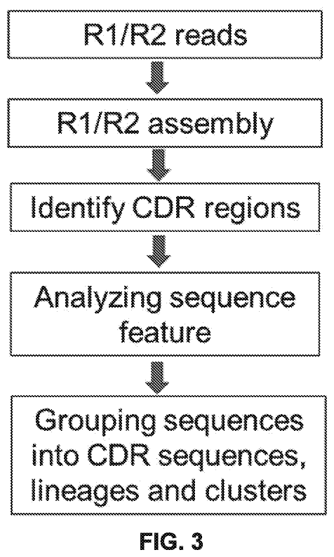

In various embodiments, at least two NGS libraries are constructed: libraries from samples before and after antigen-specific enrichment or from samples before and after immunization of the animal. Sequences generated from these libraries can be processed to identify CDR regions, germline sequence, count and frequency for each sequence (FIG. 3).

In some embodiments, an enrichment score for each sequence is generated by comparing the frequency of that sequence between two samples. Sequences can be grouped into CDR sequences if their CDR1, CDR2 and CDR3 sequences are identical. Additionally, sequences can be further grouped into lineages if sequences map to same V/J germline genes and have the same length of CDR3 with maximum one aa difference with CDR3 length longer than 4 and zero difference for CDR3 length equal or shorter than 4, and clusters if sequences have same length of CDR3 with 80% or more identity in CDR3 sequences. Similar enrichment scores for CDR groups, lineages and/or clusters are also calculated. In additional embodiments, to improve prediction results, machine learning predicted clones are further filtered based on enrichment scores in sequences, CDR groups, lineages and/or clusters. Clones that do not show any enrichment in sequences, CDR groups, lineages and/or clusters can be filtered out for testing.

In some embodiments where lineage is considered, lineage priority factors are one or more of lineages from high to low sequences abundancy, lineages from high to low amplification factor, lineages sequences abundancy change during immunization course, lineages sequences abundancy change before and after depleting certain unwanted B cells, lineages which share the same naïve B-cell origin between VHH and VH, or avoidance of developability liability sequences.

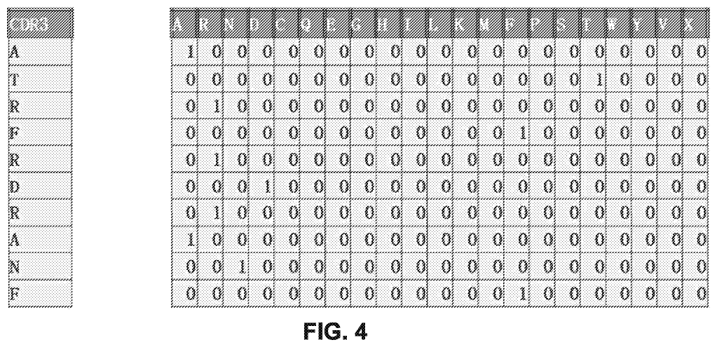

In various embodiments, sequences are converted to a matrix before feeding into machine learning model using one hot encoding (FIG. 4).

In some embodiments, input sequence can be whole antibody sequences, CDR1/2/3 sequence or CDR3 sequences. In addition, input sequences can be re-numbered and gapped based on certain scheme like IMGT so that each sequence will have same length.

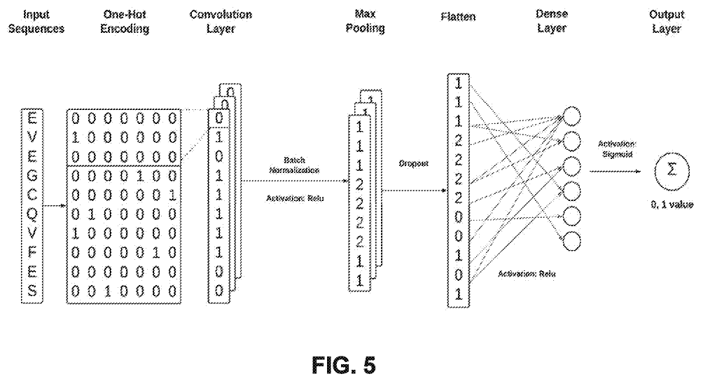

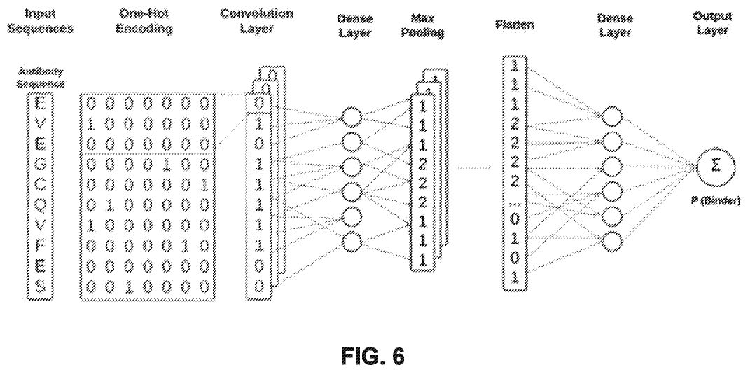

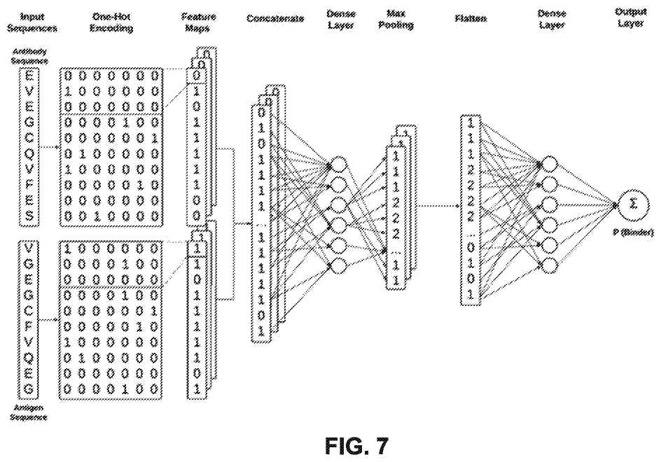

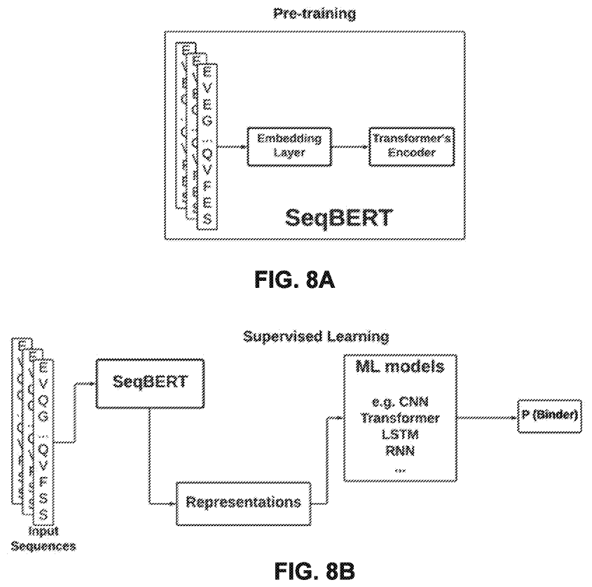

Any machine learning model algorithm or combinations of algorithms now known or later discovered can be utilized in this model. Non-limiting examples of such algorithms include a recurrent neural network (RNN), a convoluted neural network (CNN), long short-term memory (LSTM), an attention/transformer algorithm, a standard artificial neural network (ANN), a support vector machine (SVM), a random forest ensemble (RF), a decision tree (DT) model, a gaussian naïve bayes (gNB) model, a multilayer perceptron (MLP) model, a stochastic gradient descent (SGD) model, a gradient boosting (GB) model, an extreme gradient boosting (XGB) model, a light gradient boosting machine (LGB) model and logistic regression (LR) model. In some embodiments, the algorithm comprises an attention/transformer algorithm. In other embodiments, the machine learning model is built using convoluted neural network (FIG. 5, 6, 7) where binary cross-entropy is used as a loss function. Additional model parameters including number of filters, kernel size, training batch size, epochs, size of full connection neural network layer, optimizer etc. are set manually and further optimized using grid search function. In various embodiments, training data are split into two groups: high affinity binders and low affinity/nonfunctional binders based on affinity measurements, e.g., by ELISA, and used to train the model. Model performance can be measured by any means, e.g., using 10-fold cross validation. In some embodiments, an unsupervised machine learning model is developed and trained using up to millions of antibody sequences available from public domain as well as from internal sources. After the training, the machine learning model will have learned the sequences, biophysical/chemical structural features and contextual information of the input antibody sequences and generated a holistic statistical summary or mathematical representation. The weights of different variables in such trained model can be transferred to a new model for refined, supervised learning using a smaller training dataset with antibody sequences and their functional data. Alternatively the model trained with unsupervised learning can be used to convert antibody sequences in smaller training dataset to a mathematical representation, which is then used as input for further supervised learning (FIG. 8A, FIG. 8B).

Table 1 shows that the model performs well with about 90% accuracy in prediction results using training data combined from several projects or using training data from each project. In addition, the model performs similarly using whole VHH sequences, CDR sequences or CDR3 sequences as inputs. See also Example 1.

TABLE 1
Model performance measured by 10-fold cross validation

| Mean predicted accuracy | VHH | CDR | CDR3 |
|---|---|---|---|
| Total (2334 VHHs) | 0.90 | 0.90 | 0.89 |
| NBL526 only | 0.88 | 0.87 | 0.85 |
| NBL503 only | 0.92 | 0.93 | 0.94 |
| NBL509 only | 0.94 | 0.93 | 0.94 |

In additional embodiments, both antibody sequences and antigen sequences or their fragments, for example, sequences of the antibody paratopes and sequences of the antigen epitopes, are used as input to the machine learning model.

The model of the present invention can be utilized using libraries encoding any type of antibody. In some embodiments, the antibody is an immunoglobulin or fragment having two light chains and two heavy chains, or one light chain and one heavy chain. For those antibodies, the sequence used as input to the machine learning model can be heavy chain sequence only or both heavy chain and light chain sequences can be used.

In other embodiments, the antibody is a nanobody. In some of those embodiments, features that indicate an effective nanobody are evaluated by the methods described in, e.g., WO 2020/176815 and Applicant's co-pending PCT Patent Application entitled “Selection of Nanobodies Using Sequence Features” filed on Nov. 2, 2023, which are incorporated herein in their entireties. Those features include:
(a) FR2 hydrophilic region;
(b) extended CDR1;
(c) extra disulfide bond between CDR1-CDR3 or FR2-CDR3;
(d) extra disulfide bond within CDR3;
(e) long CDR3 (≥15 aa);
(f) Extra disulfide bond within CDR1;
(g) Non-classic VHH which have the same V and J germlines as conventional IgG1;
(h) Non-classic VHH which have predetermined sequence signatures;
(i) Novel canonical binding loop structure;
(j) Convergent motif or sequence signature;
(k) a phenylalanine (F) at position 42 (IMGT numbering);
(l) a short hinge;
(m) two or more cysteines in the nanobody sequence;
(n) a glutamine (Q) at position 123 (IMGT numbering);
(o) low immunogenicity metric;
(p) non-classic VHH derived from germline IGHV3;
(q) non-classic VHH derived from germline IGHV4;
(r) a histidine (H), aspartic acid (D) or glutamic acid (E) in the CDR region;
(s) a histidine (H), aspartic acid (D) or glutamic acid (E) in the first three amino acid residues, the FR2 region, or the first sixteen amino acid residues of the FR3 region of the nanobody sequence;
(t) a tyrosine (Y) at position 42 (IMGT numbering), and the nanobody having a loop, concave paratope structure configuration; or
(u) a phenylalanine (F) at position 42 (IMGT numbering), and the nanobody having a convex paratope structure configuration.

In some embodiments of the method, steps (c)-(f) are repeated to discover more sequences. In some embodiments, functions other than binding of the selected antibodies are also determined.

Any animal that produces B cells that respond to antigen can be utilized to create the model, including but not limited to a mammal (e.g., mouse, rabbit, pig, goat, camelid, etc.), a bird, a shark, etc. In a specific embodiment, the animal is a camelid.

Additionally, the model can be utilized with any type of antigen, e.g., a peptide, a protein, a hapten (e.g., conjugated to a carrier molecule), an mRNA, a DNA, a viral vector allowing the expression of an antigen of interest, or a cell.

In some embodiments, binding affinity and/or neutralizing ability of the selected antibody to the antigen and/or a second antigen is measured in steps (b). As discussed above, binding affinity can be determined by any means known in the art.

In these methods, the antibody can be expressed by any means known in the art. In some embodiments, the selected antibody is expressed in prokaryotic cells. In other embodiments, the selected antibody is expressed in eukaryotic cells.

In some embodiments of these methods, sequences predicted to have high affinity for the antigen are evaluated for development liability and eliminated for further development if the liability is present. Nonlimiting examples of development liabilities include fragmentation, immunogenicity, expression, homogeneity, solubility, stability, viscosity, and/or formulability. Some other examples of development liabilities include unpaired cysteine, N-linked glycosylation, methionine oxidation, tryptophan oxidation, asparagine deamidation, aspartic acid isomerization, lysine glycation, N-terminal glutamates, integrin binding, or CD11c/CD18 binding.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. High Affinity Nanobodies Against VEGFA Discovered by Machine Learning

Four hundred eighteen clones discovered using B cell isolation and amplification (BIA) were used as training data. ELISA affinity results of these clones were normalized to have zero average. The training data were split into two groups: 198 high affinity binders (normalized value>0) and 220 low affinity or nonfunctional binders (normalized value≤0).

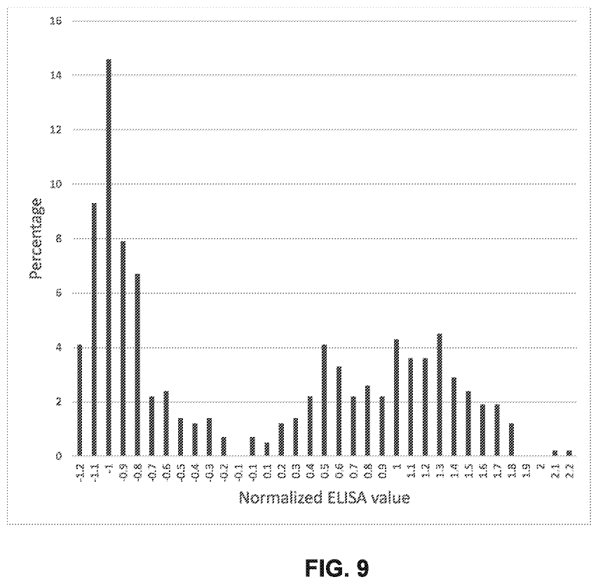

FIG. 9 shows the distribution of normalized ELISA values from training data. The result clearly shows two populations of clones in training data. A machine learning model was built as shown in FIG. 5. One hot encoded training data was used to train the model. Grid search was performed to identify best sets of parameters:

‘batch_size’: 40, ‘epochs’: 100, ‘fc_unit’: 64, ‘filters’: 16, ‘init’: ‘glorot_uniform’, ‘kernel’: 5, ‘optimizer’: ‘rmsprop’

With such parameters, the model achieved 93% accuracy in prediction based on 10-fold cross validation.

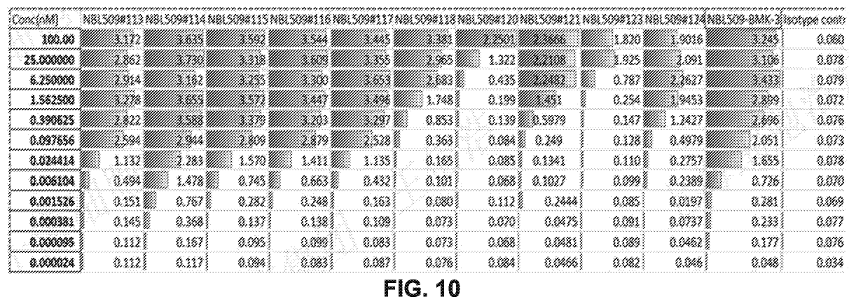

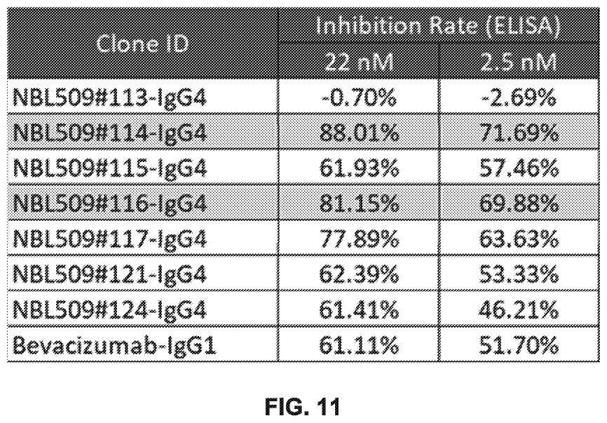

NGS sequences with count≥10 from VEGFA projects were predicted using the above trained model. For those predicted positive, 4 enrichment scores based on sequence, CDR group, lineage group and cluster group were calculated by comparing frequencies of sequences or groups in one library with those in its reference library. The reference library for a specific library is the one from samples before panning. If there is only one round panning, the reference library is the library derived from pre-panning samples. If there are two rounds of panning, then the reference library for the library from second round of panning is the one from first round of panning. Clones showing not enriched (enrichment score<2) in any of the 4 scores were filtered out. Clones with unpaired cysteine residues were further filtered out. After removing redundancy (clones with fewer than 3 residue differences in CDR regions), 13 clones were selected for synthesis and testing. ELISA results (FIG. 10) showed 10 clones are ELISA positive with a positive rate of 77%. Additional blocking assays showed that many of them have inhibition function (FIG. 11) that are higher than the inhibition activity of the benchmark bevacizumab in the blocking assays.

### Example 2. High Affinity Nanobodies Against PD-1 Discovered by Machine Learning

Six hundred forty five clones discovered using B cell isolation and amplification (BIA), phage display and clone picking from NGS data were used as training data. The training data were split into roughly equal two groups based on ELISA OD value: 311 high affinity binders (OD value>1.5) and 334 low affinity or nonfunctional binders (OD value<=1.5).

A machine learning model was built as shown in FIG. 5. One hot encoded training data was used to train the model. Grid search was performed to identify best sets of parameters:

‘batch_size’: 40, ‘epochs’: 50, ‘fc_unit’: 16, ‘filters’: 16, ‘init’: ‘normal’, ‘kernel’: 7, ‘optimizer’: ‘adam’ With such parameters, the model achieved 89% accuracy in prediction based on 10-fold cross validation.

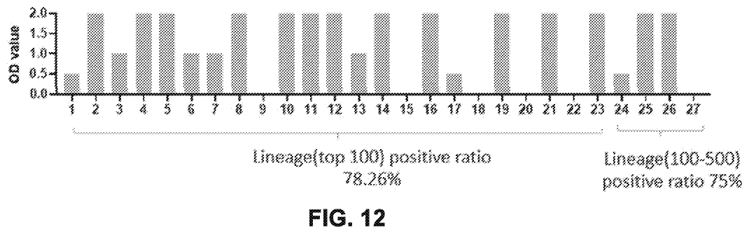

NGS sequences with count≥10 from PD-1 projects were predicted using the above trained model. For those predicted positive, 4 enrichment scores based on sequence, CDR group, lineage group and cluster group were calculated and clones showing not enriched (enrichment score<2) in any of the 4 scores were filtered out. Clones with unpaired cysteine residues were further filtered out. After removing redundancy (clones with fewer than 3 residue differences in CDR regions), 34 clones were selected for synthesis and testing. 5 of them failed to be expressed. For the rest 29 clones, 22 clones are ELISA positive with a positive rate of 76%. FIG. 12 showed lineage distribution of these 27 clones and corresponding positive rate.

### Example 3. Comparative Analysis of Machine Learning Algorithms and PD-L1 VHH Binder Discovery Using Machine Learning

One thousand and fifty seven clones with binding data identified using discovery methods like BIA, phage display and clone picking from NGS data were used as training data. The training data were split into roughly equal two groups based on ELISA OD value: 572 high affinity binders (OD value>0.6) and 485 low affinity or nonfunctional binders (OD value<=0.6).

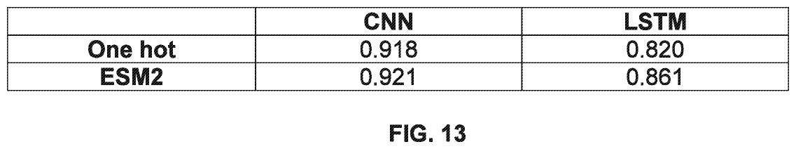

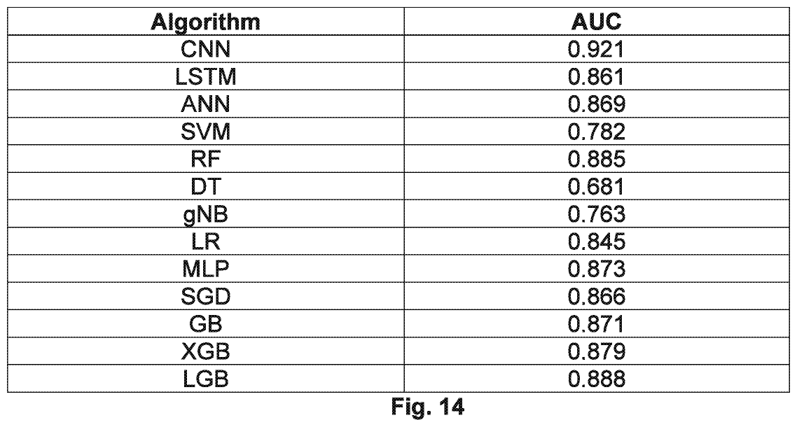

In addition to one hot encoding (FIG. 4), pre-trained model as shown in FIG. 8 was also utilized to represent sequence information. The pre-trained model we selected in this analysis is ESM2 (Lin et al., 2023). ESM-2 is a state-of-the-art protein model trained on a masked language modelling objective. It is trained on hundreds of millions of known sequences to learn possible patterns in natural proteins with billions of parameters. It is suitable for many prediction tasks using protein sequences as input (Pudžiuvelyt' et al., 2023). To compare these two sequence representation methods, we tested the performance of these two presentations using CNN and LSTM algorithms. As shown in FIG. 13, overall ESM2 representation generated better performance based on average area under curve (AUC) value from 5-fold cross validation. Using ESM2 representation, we next compared the performance of different machine learning algorithms in binder prediction. FIG. 14 shows the summary of these comparison results. Overall, CNN showed the highest average AUC value from 5-fold cross validation.

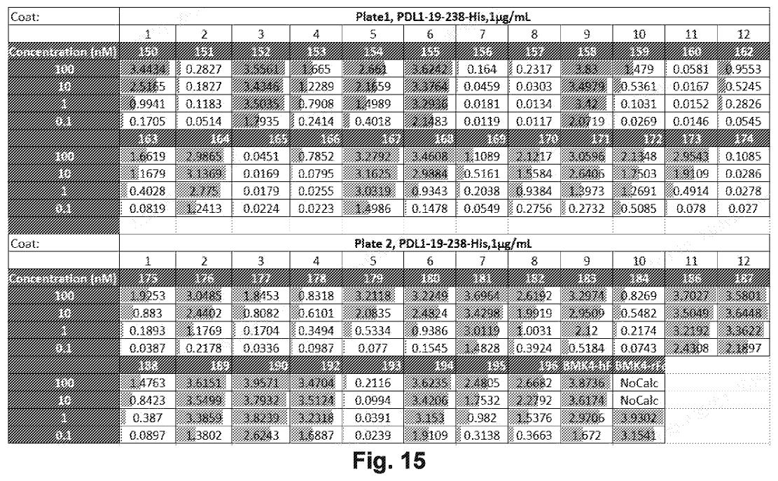

NGS sequences with count≥10 from the PD-L1 projects were predicted using the above trained model using CNN algorithm with ESM2 representation. For those predicted positive, 4 enrichment scores based on sequence, CDR group, lineage group and cluster group were calculated and clones showing not enriched (enrichment score<2) in any of 4 scores were filtered out. Clones with unpaired cysteine residues were further filtered out. After removing redundancy (clones with fewer than 5 residue differences in CDR regions), 47 clones were selected for synthesis and testing. 3 of them failed to be expressed. For the rest 44 clones, 36 clones are ELISA positive (OD>0.5 at 10 nM concentration) with a positive rate of 81%. FIG. 15 shows the ELISA binding results of these clones.

### EMBODIMENTS

Embodiment 1: A method of generating an antibody having a biological function in relation to an antigen, comprising the steps of:
a) obtaining antibodies from B cells of at least one animal immunized with the antigen;
b) determining the sequences of the antibodies in a) or fragments thereof and at least one type of functional data thereof;
c) building a machine learning model using one or more machine learning algorithms, and training the model using training data, wherein the training data comprises the sequences and functional data in b);
d) using the trained model to predict the ability of sequences from B cells of the immunized animal in a) or from B cells of a different animal immunized with the antigen, to encode an antibody that has the biological function in relation to the antigen;
e) generating one or more antibodies from the sequences in d) predicted to encode an antibody that has the biological function in relation to the antigen; and
f) determining whether the antibodies in e) has the biological function.

Embodiment 2: The method of embodiment 1, wherein the biological function is specific binding, neutralization, or potentiation.

Embodiment 3: The method of embodiment 1, wherein the functional data in b) is ELISA affinity data or neutralizing data.

Embodiment 4: The method of embodiment 1, wherein the training data in b) are generated using phage display or B cell panning.

Embodiment 5: The method of embodiment 1, wherein the sequences in b) comprise sequences of paratopes of the antibodies.

Embodiment 6: The method of embodiment 1, wherein the training data further comprises the sequences of the antigen or a portion thereof.

Embodiment 7: The method of embodiment 1, wherein the training data in b) comprises functional data of antibodies having high functional activity.

Embodiment 8: The method of embodiment 1, wherein the training data in b) comprises functional data of antibodies having low or no functional activity.

Embodiment 9: The method of any one of embodiments 1-8, wherein the machine learning is supervised.

Embodiment 10: The method of any one of embodiments 1-9, wherein the training data comprises the sequences and at least one type of functional data of few than 5,000 antibodies, 1,000 antibodies, or 500 antibodies.

Embodiment 11: The method of embodiment 1, wherein the sequences in d) are generated using high throughput sequencing technology.

Embodiment 12: The method of embodiment 11, wherein the high throughput sequencing technology is next generation sequencing technology or third generation sequencing technology.

Embodiment 13: The method of embodiment 1, wherein the machine learning algorithm is selected from a group comprising a convolutional neural network, a long short-term memory, an attention/transformer algorithm, a recurrent neural network, a standard artificial neural network, a support vector machine, a random forest ensemble, a decision tree model, a gaussian naïve bayes model, a multilayer perceptron model, a stochastic gradient descent model, a gradient boosting model, an extreme gradient boosting model, a light gradient boosting machine model, and a logistic regression model, or a combination thereof.

Embodiment 14: The method of any one of embodiments 1-13, wherein the sequences in d) predicted to encode an antibody that has the biological function in relation to the antigen are grouped by CDR groups, lineages and/or clusters.

Embodiment 15: The method of embodiment 14, wherein the sequences in a CDR group have same CDR1, CDR2 and CDR3 sequences.

Embodiment 16: The method of embodiment 14, wherein the sequences in a lineage map to the same V and J germline genes with maximum CDR3 distance of a specific CDR3 equal to or less than 1 aa between the closest two CDR3s from a lineage, wherein all CDR3s have the same length.

Embodiment 17: The method of embodiment 14, wherein the sequences in a cluster have the same CDR3 length with minimum CDR3 identity large than 80% between the closest two CDR3s from the cluster.

Embodiment 18: The method of any one of embodiments 14-17, further comprising determining enrichment scores of the sequences, CDR groups, lineages and/or clusters.

Embodiment 19: The method of embodiment 18, wherein the enrichment scores are determined by comparing the frequencies in a first library of sequences generated before antigen-specific enrichment and in a second library of sequences generated after antigen-specific enrichment.

Embodiment 20: The method of embodiment 19, wherein the enrichment scores are determined by comparing the frequencies in a first library of sequences generated from B cells before immunization of the animal and in a second library of sequences generated from B cells after immunization of the animal.

Embodiment 21: The method of any one of embodiments 18-20, wherein step e) comprises generating one or more antibodies from the sequences predicted to encode an antibody that has the biological function in relation to the antigen in d) and having a high enrichment score of the sequences.

Embodiment 22: The method of any one of embodiments 18-20, wherein step e) comprises generating one or more antibodies from the sequences predicted to encode an antibody that has the biological function in relation to the antigen in d) and having a high enrichment score of the sequences, CDR groups, lineages and/or clusters.

Embodiment 23: The method of any one of embodiments 1-22, wherein the antibody that has the biological function in relation to the antigen in d) comprises one or more of the following features:
(a) FR2 hydrophilic region
(b) extended CDR1;
(c) extra disulfide bond between CDR1-CDR3 or FR2-CDR3;
(d) extra disulfide bond within CDR3;
(e) long CDR3 (≥15 aa);
(f) Extra disulfide bond within CDR1;
(g) Non-classic VHH which have the same V and J germlines as conventional IgG1;
(h) Non-classic VHH which have predetermined sequence signatures;
(i) Novel canonical binding loop structure;
(j) Convergent motif or sequence signature;
(k) a phenylalanine (F) at position 42 (IMGT numbering);
(l) a short hinge;
(m) two or more cysteines in the nanobody sequence;
(n) a glutamine (Q) at position 123 (IMGT numbering);
(o) low immunogenicity metric;
(p) non-classic VHH derived from germline IGHV3;
(q) non-classic VHH derived from germline IGHV4;
(r) a histidine (H), aspartic acid (D) or glutamic acid (E) in the CDR region;
(s) a histidine (H), aspartic acid (D) or glutamic acid (E) in the first three amino acid residues, the FR2 region, or the first sixteen amino acid residues of the FR3 region of the nanobody sequence;
(t) a tyrosine (Y) at position 42 (IMGT numbering), and the nanobody having a loop, concave paratope structure configuration; or
(u) a phenylalanine (F) at position 42 (IMGT numbering), and the nanobody having a convex paratope structure configuration.

Embodiment 24: The method of any one of embodiments 1-23, wherein the sequences predicted to encode an antibody that has the biological function in relation to the antigen in d) are excluded if they comprise at least one development liabilities, wherein the development liabilities comprise fragmentation, immunogenicity, expression, homogeneity, solubility, stability, viscosity, and/or formulability.

Embodiment 25: The method of any one of embodiments 1-24, wherein the sequences predicted to encode an antibody that has the biological function in relation to the antigen in d) are excluded if they comprise at least one development liabilities, wherein the development liabilities is unpaired cysteine, N-linked glycosylation, methionine oxidation, tryptophan oxidation, asparagine deamidation, aspartic acid isomerization, lysine glycation, N-terminal glutamates, integrin binding, or CD11c/CD18 binding.

Embodiment 26: The method of any one of embodiments 1-25, further comprising repeating c)-f).

Embodiment 27: A method of generating an antibody that has the biological function in relation to an antigen, comprising the steps of:
a) obtaining antibodies from B cells of at least one animal immunized with the antigen;
b) determining the sequences and at least one type of functional data of the antibodies in a);
c) building a machine learning model using one or more machine learning algorithms, and training the model using training data, wherein the training data comprises the sequences and functional data of the antibodies in b);
d) using the trained model to predict the ability of sequences from B cells of the animal in a) or a different animal that is immunized with the antigen, to encode an antibody that has the biological function in relation to the antigen;
e) grouping the sequences in d) predicted to encode an antibody that has the biological function in relation to the antigen by CDR groups, lineages and/or clusters
f) determining enrichment scores of the sequences, CDR groups, lineages and/or clusters in e);
g) generating one or more antibodies from the sequences in d) predicted to encode an antibody that has the biological function in relation to the antigen and having a high enrichment score in the sequences, CDR groups, lineages and/or clusters in f); and
h) determining whether that the antibodies in g) has the biological function.

Embodiment 28: The method of embodiment 28, wherein the biological function is specific binding, neutralization, or potentiation.

Embodiment 29: The method of embodiment 27 or 28, further comprising repeating c)-h).

Embodiment 30: The method of any one of embodiments 1-29, wherein the antibody in f) is expressed by prokaryotic or eukaryotic cells.

Embodiment 31: The method of any one of embodiments 1-30, wherein an antibody is an immunoglobulin.

Embodiment 32: The method of any one of embodiments 1-30, wherein an antibody is a nanobody.

Embodiment 33: The method of any one of embodiments 1-32, wherein the animal is a mammal.

Embodiment 34: The method of embodiment 33, wherein the animal is camelid.

Embodiment 35: The method of any one of embodiments 1-34, wherein the antigen is a peptide, a protein, an mRNA, a DNA, a viral vector, and/or a cell.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

### REFERENCES

Arbabi-Ghahroudi. Camelid Single-Domain Antibodies: Historical Perspective and Future Outlook. Front. Immunol., 20 Nov. 2017.
Basilico, et al. Four individually druggable MET hotspots mediate HGF-driven tumor progression, The Journal of Clinical Investigation, Volume 124 Number 7 July, 2014.
Bird et al., Science 242:423-426 (1988).
Biswas, S., Khimulya, G., Alley, E. C., Esvelt, K. M., & Church, G. M. (2021). Low-N protein engineering with data-efficient deep learning. Nature Methods, 18(4), 389-396.
Conrath K E, et al. Emergence and evolution of functional heavy-chain antibodies in Camelidae. Dev Comp Immunol 27:87-103, 2003.
Daley L P, Clin. Vaccine Immunol. 17:239-46, 2010.
Deschacht, et al. A Novel Promiscuous Class of Camelid Single-Domain Antibody Contributes to the Antigen-Binding Repertoire, The Journal of Immunology. 184 (10) 5696-5704, May 2010.
Deschaght, P., Vintém, A. P., Logghe, M., Conde, M., Felix, D., Mensink, R., Gonçalves, J., Audiens, J., Bruynooghe, Y., Figueiredo, R., Ramos, D., Tanghe, R., Teixeira, D., van de Ven, L., Stortelers, C., & Dombrecht, B. (2017). Large diversity of functional nanobodies from a camelid immune library revealed by an alternative analysis of next-generation sequencing data. Frontiers in Immunology, 8(APR), 1-11.
Griffin et al. Analysis of heavy and light chain sequences of conventional camelid antibodies from Camelus dromedarius and Camelus bactrianus species, Journal of Immunological Methods Volume 405, Pages 35-46, March 2014.
Hollinger et al., Proc. Natl. Acad. Sci. USA, 90:6444-6448 (1993).
Huston et al., PNAS (USA) 85:5879-5883 (1988).
Klarenbeek, et al. Camelid Ig V genes reveal significant human homology not seen in therapeutic target genes, providing for a powerful therapeutic antibody platform, mAbs 7:4, 693-706; 2015.
Leem, J., Mitchell, L. S., Farmery, J. H. R., Barton, J., & Galson, J. D. (2022). Deciphering the language of antibodies using self-supervised learning. Patterns, 100513.
Lin et al., Evolutionary-scale prediction of atomic-level protein structure with a language model. Science 379, 1123-1130(2023).DOI:10.1126/science.ade2574.
Ljungars A, Svensson C, Carlsson A, Birgersson E, Tornberg U C, Frendéus B, Ohlin M, Mattsson M. Deep mining of complex antibody phage pools generated by cell panning enables discovery of rare antibodies binding new targets and epitopes. Front Pharmacol (2019) 10: doi:10.3389/fphar.2019.00847.
McCoy L E, et al. Potent and broad neutralization of HIV-1 by a llama antibody elicited by immunization. J. Exp. Med. 2012.
Narayanan, H., Dingfelder, F., Butté, A., Lorenzen, N., Sokolov, M., & Arosio, P. (2021). Machine Learning for Biologics: Opportunities for Protein Engineering, Developability, and Formulation. Trends in Pharmacological Sciences, 42(3), 151-165.
Nguyen et al. Camel heavy-chain antibodies: diverse germline VHH and specific mechanism enlarge the antigen-binding repertoire The EMBO Journal 19 No. 5 2000.
Nguyen et al. Heavy-chain antibodies in Camelidae; a case of evolutionary innovation. Immunogenetics 54:39-47, 2002.
Pudžuvelyt' I, Olechnovič K, Godliauskaite E, Sermokas K, Urbaitis T, Gasiunas G, Kazlauskas D. TemStaPro: protein thermostability prediction using sequence representations from protein language models. doi:10.1101/2023.03.27.534365.
Slatko et al, Curr. Protoc. Mol. Biol. 122:e59, 2018.
Ward et al., Nature 341, 544-546 (1989).
Woninga, et al. DNA immunization combined with scFv phage display identifies antagonistic GCGR specific antibodies and reveals new epitopes on the small extracellular loops, MABS, VOL. 8, NO. 6, 1126-1135, 2016.
Zapata et al. Protein Eng. 8(10):1057-1062 (1995).
European Patent No. 404,097.
PCT Patent Publication WO 93/11161.
PCT Patent Publication WO 2020/176815.
PCT Patent Publication WO 2020/208555.
U.S. Patent Application Publication 2019/0065677A1.
U.S. Pat. No. 5,641,870.
U.S. Pat. No. 6,054,297.
U.S. Pat. No. 5,886,152.
U.S. Pat. No. 5,877,293.

What is claimed is:

1. A method of generating an antibody having a biological function in relation to an antigen, comprising the steps of:
a) obtaining antibodies from B cells of at least one animal immunized with the antigen;
b) determining the sequences of the antibodies in a) or fragments thereof and at least one type of functional data thereof;
c) building a machine learning model using one or more machine learning algorithms, and training the model using training data, wherein the training data comprises the sequences and functional data in b);
d) using the trained model to predict the ability of sequences from B cells of the immunized animal in a) or from B cells of a different animal immunized with the antigen, to encode an antibody that has the biological function in relation to the antigen;
e) generating one or more antibodies from the sequences in d) predicted to encode an antibody that has the biological function in relation to the antigen; and
f) determining whether the antibodies in e) has the biological function.

2. The method of claim 1, wherein the biological function is specific binding, neutralization, or potentiation.

3. The method of claim 1, wherein the functional data in b) is ELISA affinity data or neutralizing data.

4. The method of claim 1, wherein the training data in b) are generated using phage display or B cell panning.

5. The method of claim 1, wherein the sequences in b) comprise sequences of paratopes of the antibodies.

6. The method of claim 1, wherein the training data further comprises the sequences of the antigen or a portion thereof.

7. The method of claim 1, wherein the training data in b) comprises functional data of antibodies having high functional activity.

8. The method of claim 1, wherein the training data in b) comprises functional data of antibodies having low or no functional activity.

9. The method of any one of claims 1-8, wherein the machine learning is supervised.

10. The method of any one of claims 1-9, wherein the training data comprises the sequences and at least one type of functional data of few than 5,000 antibodies, 1,000 antibodies, or 500 antibodies.

11. The method of claim 1, wherein the sequences in d) are generated using high throughput sequencing technology.

12. The method of claim 11, wherein the high throughput sequencing technology is next generation sequencing technology or third generation sequencing technology.

13. The method of claim 1, wherein the machine learning algorithm is selected from a group comprising a convolutional neural network, a long short-term memory, an attention/transformer algorithm, a recurrent neural network, a standard artificial neural network, a support vector machine, a random forest ensemble, a decision tree model, a gaussian naïve bayes model, a multilayer perceptron model, a stochastic gradient descent model, a gradient boosting model, an extreme gradient boosting model, a light gradient boosting machine model, and a logistic regression model, or a combination thereof.

14. The method of any one of claims 1-13, wherein the sequences in d) predicted to encode an antibody that has the biological function in relation to the antigen are grouped by CDR groups, lineages and/or clusters.

15. The method of claim 14, wherein the sequences in a CDR group have same CDR1, CDR2 and CDR3 sequences.

16. The method of claim 14, wherein the sequences in a lineage map to the same V and J germline genes with maximum CDR3 distance of a specific CDR3 equal to or less than 1 aa between the closest two CDR3s from a lineage, wherein all CDR3s have the same length.

17. The method of claim 14, wherein the sequences in a cluster have the same CDR3 length with minimum CDR3 identity large than 80% between the closest two CDR3s from the cluster.

18. The method of any one of claims 14-17, further comprising determining enrichment scores of the sequences, CDR groups, lineages and/or clusters.

19. The method of claim 18, wherein the enrichment scores are determined by comparing the frequencies in a first library of sequences generated before antigen-specific enrichment and in a second library of sequences generated after antigen-specific enrichment.

20. The method of claim 19, wherein the enrichment scores are determined by comparing the frequencies in a first library of sequences generated from B cells before immunization of the animal and in a second library of sequences generated from B cells after immunization of the animal.

21. The method of any one of claims 18-20, wherein step e) comprises generating one or more antibodies from the sequences predicted to encode an antibody that has the biological function in relation to the antigen in d) and having a high enrichment score of the sequences.

22. The method of any one of claims 18-20, wherein step e) comprises generating one or more antibodies from the sequences predicted to encode an antibody that has the biological function in relation to the antigen in d) and having a high enrichment score of the sequences, CDR groups, lineages and/or clusters.

23. The method of any one of claims 1-22, wherein the antibody that has the biological function in relation to the antigen in d) comprises one or more of the following features:
(a) FR2 hydrophilic region
(b) extended CDR1;
(c) extra disulfide bond between CDR1-CDR3 or FR2-CDR3;
(d) extra disulfide bond within CDR3;
(e) long CDR3 (≥15 aa);
(f) Extra disulfide bond within CDR1;
(g) Non-classic VHH which have the same V and J germlines as conventional IgG1;
(h) Non-classic VHH which have predetermined sequence signatures;
(i) Novel canonical binding loop structure;
(j) Convergent motif or sequence signature;
(k) a phenylalanine (F) at position 42 (IMGT numbering);
(l) a short hinge;
(m) two or more cysteines in the nanobody sequence;
(n) a glutamine (Q) at position 123 (IMGT numbering);
(o) low immunogenicity metric;
(p) non-classic VHH derived from germline IGHV3;
(q) non-classic VHH derived from germline IGHV4;
(r) a histidine (H), aspartic acid (D) or glutamic acid (E) in the CDR region;
(s) a histidine (H), aspartic acid (D) or glutamic acid (E) in the first three amino acid residues, the FR2 region, or the first sixteen amino acid residues of the FR3 region of the nanobody sequence;
(t) a tyrosine (Y) at position 42 (IMGT numbering), and the nanobody having a loop, concave paratope structure configuration; or
(u) a phenylalanine (F) at position 42 (IMGT numbering), and the nanobody having a convex paratope structure configuration.

24. The method of any one of claims 1-23, wherein the sequences predicted to encode an antibody that has the biological function in relation to the antigen in d) are excluded if they comprise at least one development liabilities, wherein the development liabilities comprise fragmentation, immunogenicity, expression, homogeneity, solubility, stability, viscosity, and/or formulability.

25. The method of any one of claims 1-24, wherein the sequences predicted to encode an antibody that has the biological function in relation to the antigen in d) are excluded if they comprise at least one development liabilities, wherein the development liabilities is unpaired cysteine, N-linked glycosylation, methionine oxidation, tryptophan oxidation, asparagine deamidation, aspartic acid isomerization, lysine glycation, N-terminal glutamates, integrin binding, or CD11c/CD18 binding.

26. The method of any one of claims 1-25, further comprising repeating c)-f).

27. A method of generating an antibody that has the biological function in relation to an antigen, comprising the steps of:
a) obtaining antibodies from B cells of at least one animal immunized with the antigen;
b) determining the sequences and at least one type of functional data of the antibodies in a);
c) building a machine learning model using one or more machine learning algorithms, and training the model using training data, wherein the training data comprises the sequences and functional data of the antibodies in b);
d) using the trained model to predict the ability of sequences from B cells of the animal in a) or a different animal that is immunized with the antigen, to encode an antibody that has the biological function in relation to the antigen;
e) grouping the sequences in d) predicted to encode an antibody that has the biological function in relation to the antigen by CDR groups, lineages and/or clusters
f) determining enrichment scores of the sequences, CDR groups, lineages and/or clusters in e);
g) generating one or more antibodies from the sequences in d) predicted to encode an antibody that has the biological function in relation to the antigen and having a high enrichment score in the sequences, CDR groups, lineages and/or clusters in f); and
h) determining whether that the antibodies in g) has the biological function.

28. The method of claim 28, wherein the biological function is specific binding, neutralization, or potentiation.

29. The method of claim 27 or 28, further comprising repeating c)-h).

30. The method of any one of claims 1-29, wherein the antibody in f) is expressed by prokaryotic or eukaryotic cells.

31. The method of any one of claims 1-30, wherein an antibody is an immunoglobulin.

32. The method of any one of claims 1-30, wherein an antibody is a nanobody.

33. The method of any one of claims 1-32, wherein the animal is a mammal.

34. The method of claim 33, wherein the animal is camelid.

35. The method of any one of claims 1-34, wherein the antigen is a peptide, a protein, an mRNA, a DNA, a viral vector, and/or a cell.
